(19) United States
(12) Patent Application Publication
(10) Pub. No.: US 2026/0130594 A1
(43) Pub. Date: May 14, 2026

(54) ELECTRONIC DEVICE FOR MONITORING PHYSIOLOGICAL SIGNALS, HEALTHCARE SYSTEM, AND RELATED METHODS

(71) Applicant: BIOPRO SCIENTIFIC CO., LTD., HSINCHU CITY (TW)

(72) Inventors: CHEN-HAN CHIEN, HSINCHU CITY (TW); HSIN-CHUAN HUANG, HSINCHU CITY (TW); TING-YUN YEN, HSINCHU CITY (TW); HSIN CHEN, HSINCHU CITY (TW)

(21) Appl. No.: 19/221,741

(22) Filed: May 29, 2025

Related U.S. Application Data
(60) Provisional application No. 63/720,449, filed on Nov. 14, 2024.

(51) Int. Cl. A61B 5/0205; A61B 5/00; A61B 5/024; A61B 5/31; A61B 5/369; G06F 40/40

(52) U.S. Cl. CPC A61B 5/0205; A61B 5/31; A61B 5/369; A61B 5/6817; A61B 5/7455; A61B 5/749; G06F 40/40; A61B 5/02416; A61B 5/02438

(57) ABSTRACT

An electronic device is provided. The electronic device includes sensing electrodes, reference electrodes, and amplifiers. The sensing electrodes are configured to obtain a first physiological signal and a second physiological signal different from the first physiological signal. The reference electrodes are configured to obtain a first reference signal and a second reference signal different from the first reference signal. The amplifiers are coupled to the plurality of sensing electrodes and the plurality of reference electrodes, and are configured to: subtract the first reference signal from the first physiological signal to generate a first data signal; subtract the second reference signal from the first physiological signal to generate a second data signal; subtract the first reference signal from the second physiological signal to generate a third data signal; and subtract the second reference signal from the second physiological signal to generate a fourth data signal.

### PRIORITY CLAIM AND CROSS-REFERENCE

The present application claims priority to U.S. Provisional Patent Application Ser. No. 63/720,449, filed on Nov. 14, 2024, which is incorporated by reference herein in its entirety.

### BACKGROUND

Electroencephalography (EEG) is widely used in cognitive state monitoring, seizure detection, sleep staging monitoring, and other neurological applications. Traditional EEG devices require a large number of electrodes arranged at various locations on the scalp, making them impractical for ambulatory use. In traditional EEG applications, one electrode among others is designated as the reference electrode, which generates the reference signal (i.e., the reference channel). The reference signal is subtracted from each of the signals from the other electrodes to obtain data signals (i.e., the data channels). However, the single reference electrode limits the number of usable data signals. Moreover, signals from electrodes near the reference electrode may be similar to the reference signal in waveform and/or amplitude, causing signal quality of the data signals to deteriorate.

### SUMMARY

It is one aspect of the present disclosure to provide an electronic device. The electronic device includes a plurality of sensing electrodes, a plurality of reference electrodes, and a plurality of amplifiers. The plurality of sensing electrodes are configured to obtain a first physiological signal and a second physiological signal different from the first physiological signal. The plurality of reference electrodes are configured to obtain a first reference signal and a second reference signal different from the first reference signal. The plurality of amplifiers are coupled to the plurality of sensing electrodes and the plurality of reference electrodes, and are configured to: subtract the first reference signal from the first physiological signal to generate a first data signal; subtract the second reference signal from the first physiological signal to generate a second data signal; subtract the first reference signal from the second physiological signal to generate a third data signal; and subtract the second reference signal from the second physiological signal to generate a fourth data signal.

It is another aspect of the present disclosure to provide an electronic device. The electronic device includes a pair of sensing electrodes, a pair of reference electrodes, and a controller. The pair of sensing electrodes are configured to obtain a first physiological signal and a second physiological signal corresponding to outer ear canals of a user. The pair of reference electrodes are configured to obtain a first reference signal and a second reference signal corresponding to mastoid processes of the user. The controller is coupled to the pair of sensing electrodes and the pair of reference electrodes, and is configured to generate an analysis result according to (1) differences between the first physiological signal and each of the first reference signal and the second reference signal and (2) differences between the second physiological signal and each of the first reference signal and the second reference signal.

It is yet another aspect of the present disclosure to provide a method for monitoring physiological signals. The method includes the operations: obtaining a first physiological signal and a second physiological signal different from the first physiological signal; obtaining a first reference signal and a second reference signal different from the first reference signal; and generating an analysis result by (1) comparing the first physiological signal with each of the first reference signal and the second reference signal and (2) comparing the second physiological signal with each of the first reference signal and the second reference signal. The first physiological signal and the second physiological signal are obtained by a pair of sensing electrodes arranged on opposite sides of scalp. The first reference signal and the second reference signal are obtained by a pair of reference electrodes arranged on opposite sides of scalp.

It is yet another aspect of the present disclosure to provide a method for processing physiological data. The method includes the operations: using the above mentioned electronic device (i.e., the electronic device having the pair of sensing electrodes, the pair of reference electrodes, and the controller) to obtain the analysis result; receiving a natural language input (prompt) to a machine learning model; generating a natural language output by processing the analysis result and the natural language input by using the machine learning model; and providing the natural language output. The analysis result includes at least one of a physiological data selected from a group consisting of power of brain waves of different frequency bands, asymmetry of brain waves of a predetermined frequency band, an arousal level, and an emotional valence.

It is yet another aspect of the present disclosure to provide a healthcare system. The healthcare system includes an electronic device mentioned above and a cloud server. The electronic device is configured to obtain the analysis result. The cloud server is configured to execute a machine learning model. The cloud server is further configured to: receive a natural language input to the machine learning model, wherein the natural language input indicates a role-playing scenario; generate a natural language output by processing the analysis result and the natural language input by using the machine learning model; and provide the natural language output. The analysis result includes at least one of a physiological data selected from a group consisting of power of brain waves of different frequency bands, asymmetry of brain waves of a predetermined frequency band, an arousal level, and an emotional valence.

The electronic device and method mentioned above reduce the amount of required electrodes to be positioned on the scalp, and also prevent the signal quality deterioration caused by the physiological signals being too close to the reference signals.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a circuit schematic diagram of a signal processing circuit according to some embodiments of the present disclosure.

FIG. 2A is a circuit schematic diagram of a signal processing circuit according to some embodiments of the present disclosure.

FIG. 2B is a circuit schematic diagram of a signal processing circuit according to some embodiments of the present disclosure.

FIG. 3A is a schematic diagram illustrating the generation of data signals performed by a comparative EEG device with eight sensing electrodes and a reference electrode.

FIG. 3B is a schematic diagram illustrating the generation of data signals performed by the signal processing circuit shown in FIG. 2B with six sensing electrodes and two reference electrodes, according to some embodiments of the present disclosure.

FIG. 3C is a schematic diagram showing positions of the scalp suitable for placing the reference electrodes of the signal processing circuit, according to some embodiments of the present disclosure.

FIG. 4 is a circuit schematic diagram of a signal processing circuit according to some embodiments of the present disclosure.

FIG. 5 is a schematic block diagram of an electronic device according to some embodiments of the present disclosure.

FIG. 6 is a flowchart diagram of a method performed by the electronic device for monitoring physiological signals, according to some embodiments of the present disclosure.

FIG. 7A is for illustrating correlation between data signals obtained respectively by the electronic device according to some embodiments of the present disclosure and the EEG device according to some comparative embodiments.

FIG. 7B is for illustrating correlation between data signals obtained respectively by the electronic device according to some embodiments of the present disclosure and the EEG device according to some comparative embodiments.

FIG. 8 shows a perspective view of the electronic device according to some embodiments of the present disclosure.

FIG. 9 is a schematic functional block diagram of the healthcare system according to some embodiments of the present disclosure.

FIG. 10 is a flowchart of a method for processing physiological data performed by the healthcare system of FIG. 9, according to some embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the present disclosure may repeat reference numerals and/or letters in the various examples. This repetition is for the purpose of simplicity and clarity and does not in itself dictate a relationship between the various embodiments and/or configurations discussed.

Spatially relative terms, such as “beneath,” “below,” “lower,” “above,” “upper” and the like, may be used herein for ease of description to describe one element or feature's relationship to another element(s) or feature(s) as illustrated in the drawings. The spatially relative terms are intended to encompass different orientations of the device in use or operation in addition to the orientation depicted in the drawings. The apparatus may be otherwise oriented (e.g., rotated by 90 degrees or at other orientations) and the spatially relative descriptors used herein may likewise be interpreted accordingly. Further, it will be understood that when an element is referred to as being “connected to” or “coupled to” another element, it may be directly connected to or coupled to the other element, or intervening elements may be present.

The burden of mental health issues has significant economic implications on a global scale. These losses primarily arise from decreased productivity, healthcare expenses, and social service costs. The impact on labor markets is particularly severe, with depression and anxiety leading to significant annual losses worldwide, excluding the associated healthcare and social service costs. These factors collectively create an unprecedented global burden in addressing mental health issues.

Despite this significant impact, current treatment options remain limited. The standard treatment protocol begins with medication and psychotherapy or counseling. For those who encounter drug-resistant conditions, neuromodulation techniques such as cranial electrical stimulation, transcranial magnetic stimulation, and vagus nerve stimulation are considered. However, commonly used medications have wide-ranging effects on the body, and their strong side effects often necessitate several clinical visits to determine the most suitable option for each patient. This adjustment period typically spans three to six months, creating substantial challenges for patients in need of immediate relief. Commonly prescribed medications include selective serotonin reuptake inhibitors (SSRIs), serotonin norepinephrine reuptake inhibitors (SNRIs), norepinephrine dopamine reuptake inhibitors (NDRIs), monoamine oxidase inhibitors (MAOIs), tricyclics, and triptans.

Noninvasive neuromodulation usually comes into consideration when medication fails to improve the situation. These neuromodulation treatments have shown promising effects in helping mental health patients, with significantly fewer side effects compared to medication and generally better outcomes. The primary reason for not employing neuromodulation as a first-line treatment lies in the lack of knowledge and accessibility, which is largely related to the high cost of neuromodulation devices compared to medications.

Given the long history of mental health drug development and the continuing unmet needs, the World Health Organization has extended its mental health action plan. This plan advocates for a more robust support system in society while highlighting the urgent need for innovative technologies and approaches in mental health treatment.

Various EEG features can be identified as potential biomarkers for mental illness and cognitive dysfunction. A notable example is alpha asymmetry in the frontal lobe, which has frequently been associated with depression and anxiety. However, emerging evidence suggests that this relationship is more complex than initially thought; for example, alpha asymmetry patterns are gender-dependent and may serve as predictors of drug responsiveness. Therefore, there are opportunities to use EEG signals to categorize patients with mental disorders, such as those with chronic migraines or anxiety. Collectively, EEG signals could play a crucial role in personalizing treatment strategies—from medication selection and neuromodulation method choice to optimizing stimulation parameters and monitoring treatment efficacy.

Accordingly, there is a need for accessible personalized physiological monitoring and neuromodulation systems, which can manage mental health disorders and increase overall wellness by providing an integrated solution for continuous assessment, personalized intervention, and collaborative care between individuals and healthcare professionals.

In some embodiments of the present disclosure, the electronic device may utilize sensors of multiple types at different locations, along with edge computing power, to enhance the capacity for personalized neuromodulation. In some embodiments, the device may incorporate cloud-based AI for further analysis, improving the precision of personalized health monitoring. Additionally, the cloud-based AI may provide suggestions for general wellness management, and may act as a practitioner of EEG neurofeedback (biofeedback) who teaches self-control of brain (physiological) functions to subjects.

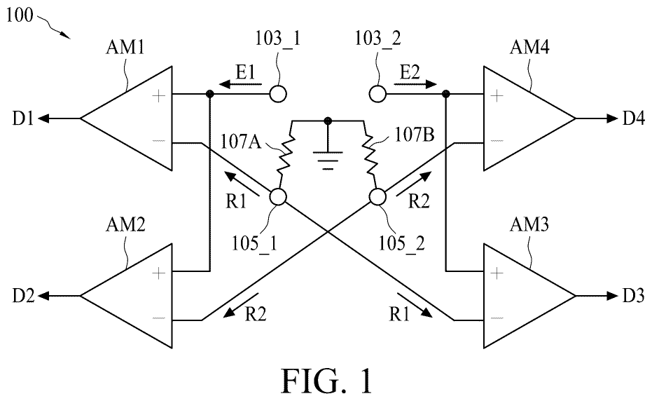

FIG. 1 is a circuit schematic diagram of a signal processing circuit 100 according to some embodiments of the present disclosure. The signal processing circuit 100 includes amplifiers AM1 to AM4. The amplifiers AM1 to AM4 may be coupled to sensing electrodes 103_1 and 103_2 and reference electrodes 105_1 and 105_2, so as to receive physiological signals E1 and E2 and reference signals R1 and R2. The signal processing circuit 100 is configured to subtract the reference signals R1 and R2 from the physiological signals E1 and E2, which will be explained in conjunction with the circuit structure of the signal processing circuit 100.

A non-inverting input terminal of the amplifier AM1 and a non-inverting input terminal of the amplifier AM2 are coupled to each other, and are configured to receive the physiological signal E1 from the sensing electrode 103_1. An inverting input terminal of the amplifier AM1 is configured to receive the reference signal R1 from the reference electrode 105_1, while an inverting input terminal of the amplifier AM2 is configure to receive the reference signal R2 from the reference electrode 105_2. Therefore, the amplifier AM1 is configured to subtract the reference signal R1 from the physiological signal E1 to obtain a data signal D1 (i.e., D1=E1−R1); and the amplifier AM2 is configured to subtract the reference signal R2 from the physiological signal E1 to obtain a data signal D2 (i.e., D2=E1-R2).

A non-inverting input terminal of the amplifier AM3 and a non-inverting input terminal of the amplifier AM4 are coupled to each other, and are configured to receive the physiological signal E2 from the sensing electrode 103_2. An inverting input terminal of the amplifier AM3 is configured to receive the reference signal R1 from the reference electrode 105_1, while an inverting input terminal of the amplifier AM4 is configure to receive the reference signal R2 from the reference electrode 105_2. Therefore, the amplifier AM3 is configured to subtract the reference signal R1 from the physiological signal E2 to obtain a data signal D3 (i.e., D3=E2−R1); and the amplifier AM4 is configured to subtract the reference signal R2 from the physiological signal E2 to obtain a data signal D4 (i.e., D4=E2−R2). In some embodiments, the inverting input terminals of the amplifiers AM1 and AM3 are coupled together and further coupled to the reference electrode 105_1; and the inverting input terminals of the amplifiers AM2 and AM4 are coupled together and further coupled to the reference electrode 105_2.

In some embodiments, the signal processing circuit 100 further includes resistors 107A and 107B for providing bias voltages. A first terminal of the resistor 107A is coupled to the inverting input terminals of the amplifiers AM1 and AM3 through the reference electrode 105_1. A first terminal of the resistor 107B is coupled to the inverting input terminals of the amplifiers AM2 and AM4 through the reference electrode 105_2. Second terminals of the resistors 107A and 107B are coupled to a ground terminal. The ground terminal may be an electrode of a battery driving the signal processing circuit 100. The resistance of the resistors 107A and 107B may be set equal and within a range of about 0.1 kΩ to about 10 MΩ.

The physiological signals E1 to E2 and the reference signals R1 and R2 of FIG. 1 may be electroencephalography (EEG) signals. In some embodiments, the physiological signals E1 and E2 correspond to left and right outer ear canals of a user, respectively; the reference signals R1 and R2 correspond to left and right mastoid processes of the user, respectively.

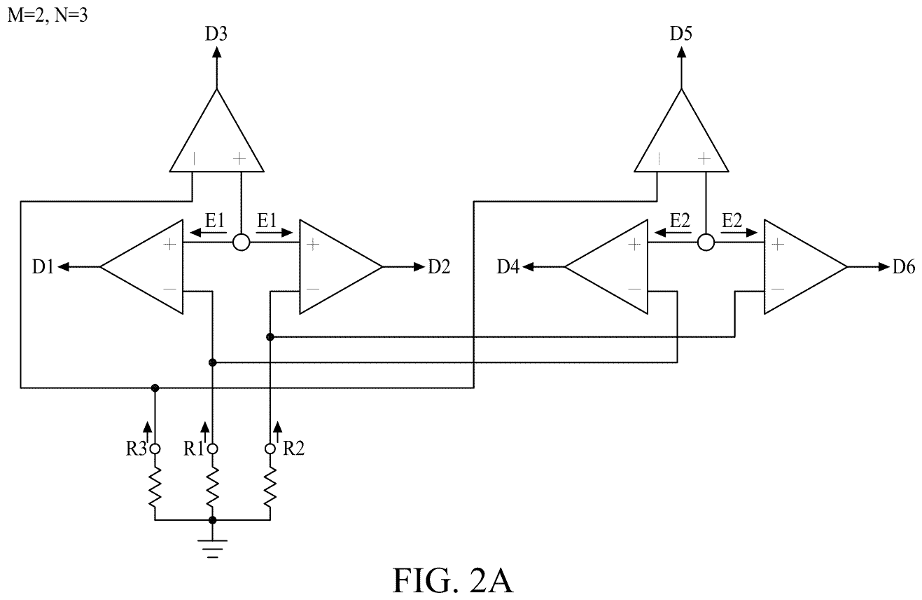

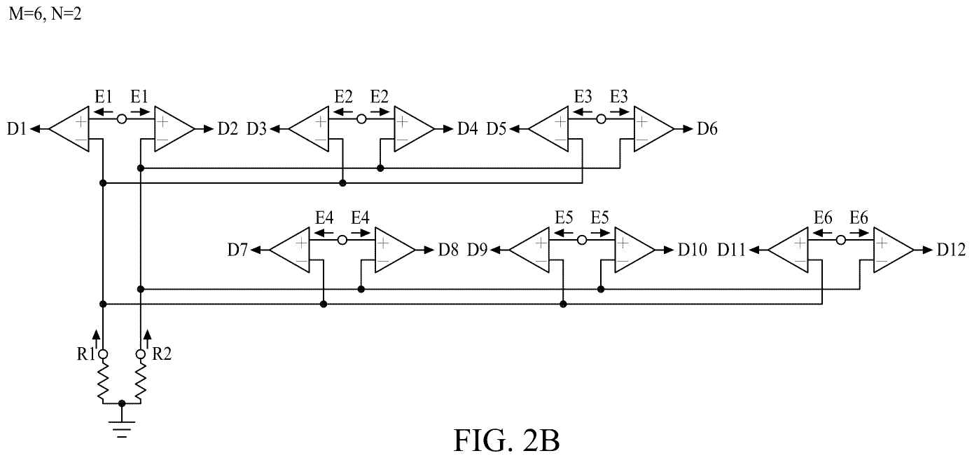

The signal processing circuit 100 of FIG. 1 can be expanded to obtain more data signals. The manner for expanding the signal processing circuit is illustrated in greater details with reference to FIGS. 2A and 2B, where FIGS. 2A and 2B are circuit schematic diagrams of various signal processing circuits according to some embodiments of the present disclosure. By respectively subtracting the reference signals from each physiological signal, the signal processing circuits of FIGS. 2A and 2B can obtain M×N EEG data signals from M physiological signals and N reference signals, where M and N are positive integers. Alternatively stated, each of the signal processing circuits of FIGS. 2A and 2B include M×N amplifiers when the number of sensing electrodes is M and the number of reference electrodes is N. The number of the resistors is equal to the number of the reference electrodes, and the reference electrodes are coupled to the ground terminal through these resistors, respectively. In the embodiment of FIG. 2A that M is two (2) and N is three (3), the data signals D1 to D6 are generated by respectively subtracting the reference signals R1, R2, and R3 from each of the physiological signals E1 and E2. In another embodiment of FIG. 2B that M is six (6) and N is two (2), the data signals D1 to D12 are generated by respectively subtracting the reference signals R1 and R2 from each of the physiological signals E1 to E6.

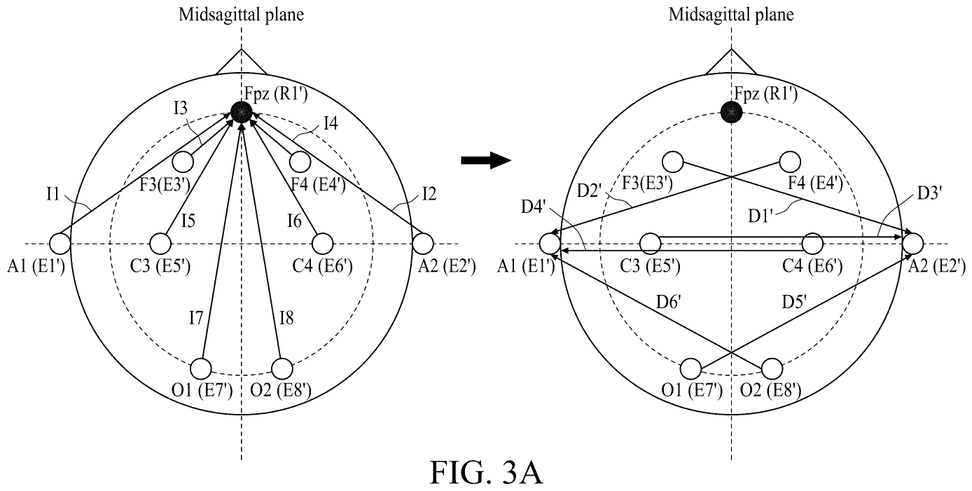

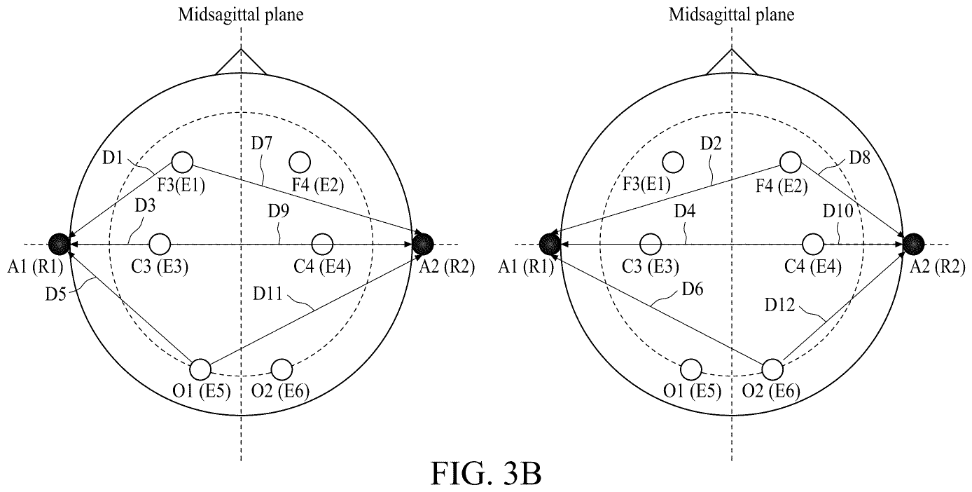

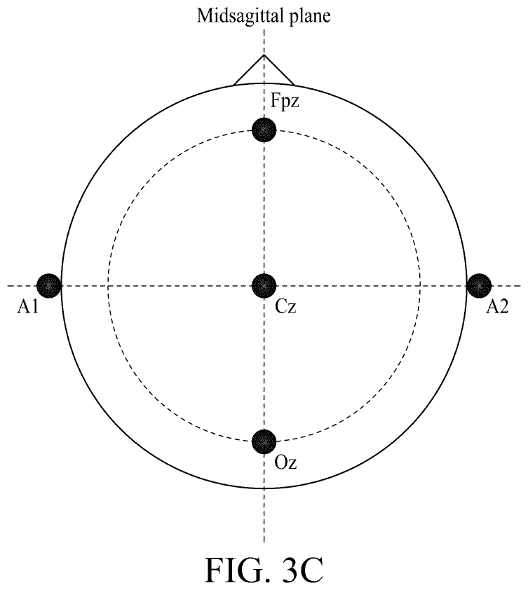

The signal processing circuits of the above embodiments of FIGS. 1, 2A, and 2B can reduce the amount of required electrodes to be positioned on the scalp, and are free from complicated software based calculations to convert the physiological signals to suitable data signals, which will be explained with reference to FIG. 3A, FIG. 3B, and FIG. 3C. FIG. 3A is a schematic diagram illustrating the generation of data signals performed by a comparative EEG device with eight (8) sensing electrodes and a reference electrode. In the circumstance of determining the physiological status (e.g., the sleeping stage) of a user, the comparative EEG device may be configured to obtain eight (8) physiological signals E1′ to E8′ and a reference signal R1′. The physiological signals E1′ and E2′ are obtained from the positions “A1” and “A2” (e.g., the regions of scalp corresponding to the mastoid processes) in the 10-20 system. The physiological signals E3′ and E4′ are obtained from the positions “F3” and “F4” (e.g., the regions of scalp corresponding to the frontal lobes) in the 10-20 system. The physiological signals E5′ and E6′ are obtained from the positions “C3” and “C4” (e.g., the regions corresponding to the center of the scalp) in the 10-20 system. The physiological signals E7′ and E8′ are obtained from the positions “O1” and “O2” (e.g., the regions of scalp corresponding to the occipital lobes) in the 10-20 system. The reference signal R1′ is obtained from the position “Fpz” in the 10-20 system. The position Fpz is the region of the scalp that is on the midsagittal plane and corresponding to the pre-frontal lobes. The comparative EEG device has to subtract the reference signal R1 from each of the physiological signals E1′ to E8′ to obtain multiple intermediate signals I1 to I8, which may be represented as E1′-R1′, E2′-R1′, E3′-R1′, E4′-R1′, E5′-R1′, E6′-R1′, E7′-R1′, and E8′-R1′, respectively. However, these intermediate signals are not recommended for the determination of physiological sleep status. The position “Fpz” is near the eyes; therefore, the EEG signals using the position “Fpz” as the reference point are susceptible to contamination by eye movements. Therefore, the comparative EEG device has to further subtract the intermediate data signals I1 and I2 from other intermediate data signals I3 to I8 to obtain six (6) data signals D1′ to D6′ suitable for the determination of physiological status, which are shown in TABLE I and FIG. 3A. It is noted that the comparative EEG device relies on time consuming software based calculations to obtain the data signals D1′ to D6′.

TABLE I

| Data signal | |
|---|---|
| D1′ | E3′-E2′ (F3-A2) |
| D2′ | E4′-E1′ (F4-A1) |
| D3′ | E5′-E2′ (C3-A2) |
| D4′ | E6′-E1′ (C4-A1) |
| D5′ | E7′-E2′ (O1-A2) |
| D6′ | E8′-E1′ (O2-A1) |

FIG. 3B is a schematic diagram illustrating the generation of data signals performed by the signal processing circuit shown in FIG. 2B with six sensing electrodes (i.e., M=6) and two reference electrodes (i.e., N=2), according to some embodiments of the present disclosure. In the circumstance of determining the physiological status of the user, the signal processing circuit may be configured to obtain six (6) physiological signals E1 to E6 and two (2) reference signals R1 and R2. The physiological signals E1 and E2 are obtained from the positions “F3” and “F4” in the 10-20 system. The physiological signals E3 and E4 are obtained from the positions “C3” and “C4” in the 10-20 system. The physiological signals E5 and E6 are obtained from the positions “O1” and “O2” in the 10-20 system. The reference signals R1 and R2 are obtained from the positions “A1” and “A2” in the 10-20 system. As shown in FIG. 2B, the reference signals R1 and R2 may be rapidly subtracted from the physiological signals E1 to E6 by the amplifiers, so as to obtain the twelve (12) data signals D1 to D12 shown in TABLE II and FIG. 3B.

TABLE II

| Data signal | |
|---|---|
| D1 | E1-R1 (F3-A1) |
| D2 | E2-R1 (F4-A1) |
| D3 | E3-R1 (C3-A1) |
| D4 | E4-R1 (C4-A1) |
| D5 | E5-R1 (O1-A1) |
| D6 | E6-R1 (O2-A1) |
| D7 | E1-R2 (F3-A2) |
| D8 | E2-R2 (F4-A2) |
| D9 | E3-R2 (C3-A2) |
| D10 | E4-R2 (C4-A2) |
| D11 | E5-R2 (O1-A2) |
| D12 | E6-R2 (O2-A2) |

The data signals D2, D4, D6, D7, D9, and D11 can reflect the activities of different brain regions on the opposite sides of the scalp defined by the midsagittal plane. Therefore, the data signals D2, D4, D6, D7, D9, and D11 are suitable for the determination of physiological status. The data signals D1, D3, D5, D8, D10, and D12 can reflect the activities of different brain regions on the same side of the scalp defined by the midsagittal plane. The data signals D1, D3, D5, D8, D10, and D12 may be used to improve the precision of the determination of physiological status. It is noted that, under similar montages (i.e., the distributions of the electrodes), the signal processing circuit of FIG. 2B can generate data signals much more than that of the comparative EEG device in a short time, as the signal processing circuit of FIG. 2B is free from complicated software based calculations to convert the physiological signals to suitable data signals.

FIG. 3C is a schematic diagram showing positions of the scalp suitable for placing the reference electrodes of the signal processing circuit, according to some embodiments of the present disclosure. The reference electrodes of the signal processing circuits of the present disclosure may be placed on at least some of the positions Fpz, Cz, Oz, A1, and A2 of the 10-20 system, while the sensing electrodes of the signal processing circuits of the present disclosure may be placed on the other positions of the 10-20 system. The positions Fpz, Cz, and Oz are on the midsagittal plane and respectively corresponding to the frontal lobes, the center of the scalp, and the occipital lobes. Since the brain region corresponding to the position Cz receives lots of attention in the determination of physiological status, placing the reference electrode at the position Cz can eliminate the software based calculations to convert the physiological signals to data signals, similar to the advantages of placing the reference electrodes at the positions A1 and A2.

The physiological signals from sensing electrodes distant from the reference electrode may have waveform and/or amplitude significantly different from the reference signal. The divergence between the physiological signals and reference signals may improve the signal quality of data signals. Therefore, placing the reference electrode at the position Fpz may improve the signal quality of data signals regarding the occipital lobes; and placing the reference electrode at the position Oz may improve the signal quality of data signals regarding the frontal lobes.

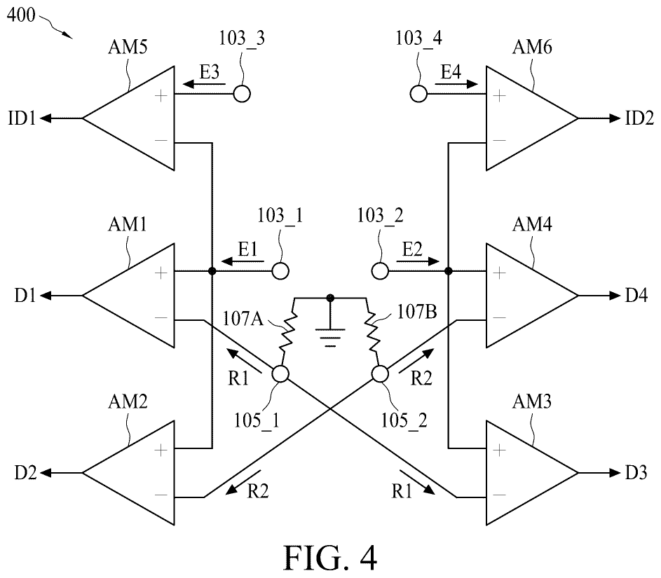

In some embodiments, software-based signal processing may collaborate with the signal processing circuits of the present disclosure to realize expansion of data signals, so as to reduce the number of amplifiers. FIG. 4 is a circuit schematic diagram of a signal processing circuit 400 according to some embodiments of the present disclosure. The signal processing circuits 100 and 400 have similarities; therefore, only the differences between them are described in the following paragraphs. The signal processing circuit 400 includes amplifiers AM5 and AM6. A non-inverting input terminal of the amplifier AM5 is configured to receive a physiological signal E3 from a sensing electrode 103_3. An inverting input terminal of the amplifier AM5 is coupled to the non-inverting input terminal of the amplifier AM1 to receive the physiological signal E1. Therefore, the amplifier AM5 is configured to subtract the physiological signal E1 from the physiological signal E3 to obtain an intermediate data signal ID1 (i.e., ID1=E3−E1). A non-inverting input terminal of the amplifier AM6 is configured to receive a physiological signal E4 from a sensing electrode 103_4. An inverting input terminal of the amplifier AM6 is coupled to the non-inverting input terminal of the amplifier AM4 to receive the physiological signal E2. Therefore, the amplifier AM6 is configured to subtract the physiological signal E2 from the physiological signal E4 to obtain an intermediate data signal ID2 (i.e., ID2=E4−E2). In some embodiments, the physiological signals E3 and E4 are EEG signals corresponding to left and right forehead lobes of the user, respectively.

The signal processing circuit 400 may be configured to perform data signal expansion according to the data signals D1 to D4 and the intermediate data signals ID1 and ID2. For example, the controller may add the intermediate data signal ID1 to the data signal D1 to obtain a first additional data signal (i.e., E3-R1); the controller may add the intermediate data signal ID1 to the data signal D2 to obtain a second additional data signal (i.e., E3-R2); the controller may add the intermediate data signal ID2 to the data signal D3 to obtain a third additional data signal (i.e., E4-R1); and the controller may add the intermediate data signal ID2 to the data signal D4 to obtain a fourth additional data signal (i.e., E4-R2).

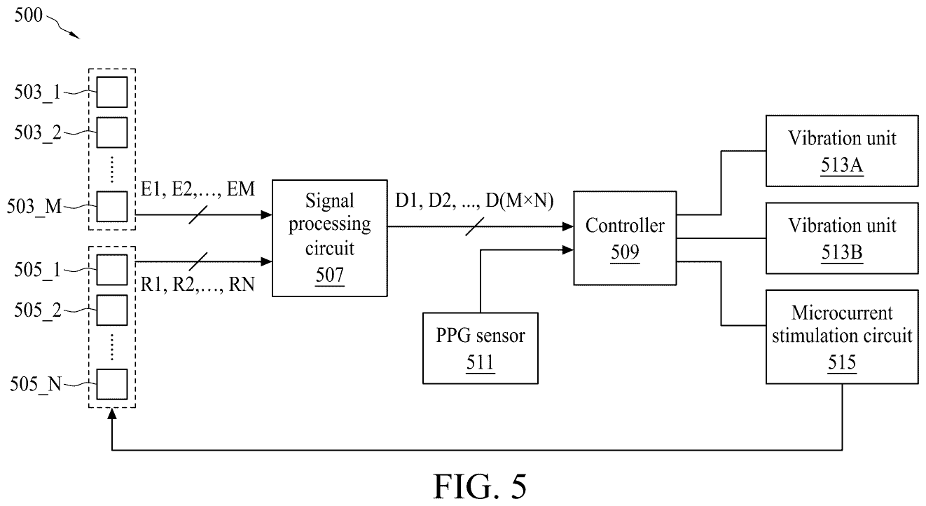

FIG. 5 is a schematic block diagram of an electronic device 500 according to some embodiments of the present disclosure. The electronic device 500 includes sensing electrodes 503_1 to 503_M, reference electrodes 505_1 to 505_N, a signal processing circuit 507, and a controller 509. The signal processing circuit 507 is configured to receive the physiological signals E1 to EM and the reference signals R1 to RN from the sensing electrodes 503_1 to 503_M and the reference electrodes 505_1 to 505_N. For the purpose of facilitating understanding, the electronic device 500 is described in the following paragraphs under the condition that M and N are both two (2), where under this condition the signal processing circuit 507 may be realized with the signal processing circuit 100 of FIG. 1. However, the number of the sensing electrodes and the reference electrodes are provided for illustrative purposes only, and the present disclosure is not limited thereto. For example, the electronic device 500 may be realized by the signal processing circuit of FIG. 2A, FIG. 2B, or FIG. 4 in some embodiments.

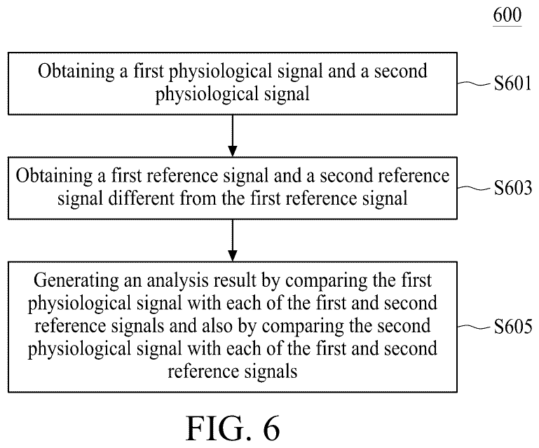

The electronic device 500 may be a wearable device, and configured to generate an analysis result of a user physiological status. FIG. 6 is a flowchart diagram of a method 600 performed by the electronic device 500 for monitoring the physiological signals, according to some embodiments of the present disclosure. Referring to FIG. 5 and FIG. 6, in operation S601, the sensing electrodes 503_1 and 503_2 obtain the physiological signals E1 and E2, respectively. The physiological signals E1 and E2 are different from each other. The sensing electrodes 503_1 and 503_2 may be a pair of electrodes that are arranged on opposite sides of the scalp, for example, in the left and right outer car canals of the user; however, the present disclosure is not limited thereto.

In operation S603, the reference electrodes 505_1 and 505_2 obtain the reference signals R1 and R2, respectively. The reference signals R1 and R2 are different from each other. The reference electrodes 505_1 and 505_2 may be a pair of electrodes that are arranged on opposite sides of the scalp, for example, at the left and right mastoid processes of the user; however, the present disclosure is not limited thereto.

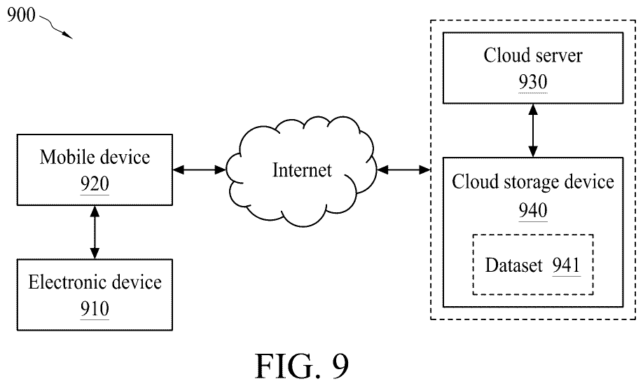

In operation S605, the analysis result of the user physiological status is generate by (1) comparing the physiological signal E1 with each of the reference signals R1 and R2 and (2) comparing the physiological signal E2 with each of the reference signals R1 and R2. Specifically, the signal processing circuit 507 generates the data signals D1 to D4, where D1=E1−R1; D2=E1−R2; D3=E2−R1; and D4=E2−R2. The controller 509 receives the data signals D1 to D4, and generates the analysis result according to the data signals D1 to D4. The controller 509 may further determining a sleep stage, the emotional status, and/or the focus level of the user based on the analysis result, as the data signals D1 to D4 are generated based on signals measured on opposite sides of the scalp defined by the midsagittal plane and signals measured on the same side of the scalp defined by the midsagittal plane; however, this disclosure is not limited thereto. In some embodiments, the controller 509 may transmit the analysis result to a mobile device 920 in FIG. 9 for the mobile device 920 to determining the sleep stage, the emotional status, and/or the focus level of the user based on the analysis result. In some embodiments that the signal analysis circuit 507 is realized by the signal analysis circuit 400 of FIG. 4, the controller 509 may generate the analysis result according to not only the data signals D1 to D4 but also the first through fourth additional data signals derived from the intermediate data signals ID1 and ID2.

Referring to FIG. 5 again, the electronic device 500 further includes a photoplethysmography (PPG) sensor 511, vibration units 513A and 513B, and a microcurrent stimulation circuit 515 that are coupled to the controller 509. The PPG sensor 511 is configured to obtain a PPG signal corresponding to one of the mastoid processes of the user; however, the present disclosure is not limited thereto. In some embodiments, the PPG signal may be obtained from an area of the scalp other than or near the mastoid processes. The controller 509 may generates the analysis result according to the PPG signal and the data signals D1 to D4. The controller 509 may control the vibration intensities, waveform, and/or frequencies of the vibration units 513A and 513B according to the analysis result or user inputs from, for example, the mobile device 920 in FIG. 9. Furthermore, the controller 509 may control the amplitude and frequency of the currents outputted from the microcurrent stimulation circuit 515 to the reference electrodes 505_1 to 505_N. In other words, the reference electrodes 505_1 to 505_N are not only configured to obtain reference signals R1 to RN, but are also configured to output stimulation currents; however, the present disclosure is not limited thereto. In some embodiments, the microcurrent stimulation circuit 515 may output the currents to one or more electrodes other than the reference electrodes 505_1 to 505_N. Therefore, the electronic device 500 can provide neuromodulation actions such as vibration and microcurrent stimulation, in order to promote relaxation and/or focus level of the user. In some embodiments, the electronic device 500 further includes a motion sensor, a microphone, and near-infrared (NIR) LEDs, where the motion sensor and the microphone are configured to capture the motion data and the audio data of the user. The controller 509 may generates the analysis result according to a motional signal outputted by the motion sensor, the PPG signals, and the data signals D1 to D4.

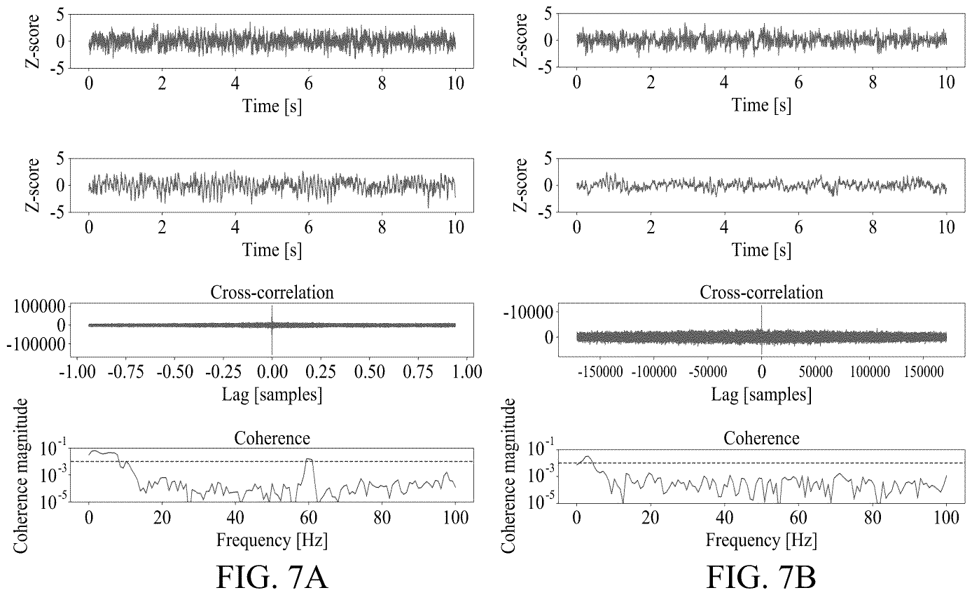

FIG. 7A is for illustrating correlation between data signals obtained respectively by the electronic device 500, according to some embodiments of the present disclosure, and the EEG device, according to some comparative embodiments (hereinafter “comparative EEG device”). The first row of FIG. 7A is the waveform of the data signal D2, which is E1-R2, generated by the electronic device 500. The second row of FIG. 7A is the waveform of the comparative C-type signal generated by the comparative EEG device. The comparative C-type signal may be generated by subtracting an EEG signal obtained from the position “C3” of the 10-20 system from another EEG signal obtained from the position “A2” of the 10-20 system. The third row of FIG. 7A is the cross-correlation plot of the data signal D2 and the comparative C-type signal. The cross-correlation plot peaks at lag 0, which indicates that the data signal D2 and the comparative C-type signal have strong relationship in the time domain. The fourth row of FIG. 7A is the coherence plot of the data signal D2 and the comparative C-type signal. With respect to the low frequency region (e.g., 0-5 Hz) focused by the polysomnography studies, the coherence magnitude is about 5×10⁻², which indicates that the data signal D2 and the comparative C-type signal have a good relationship in low frequencies.

FIG. 7B is for illustrating correlation between data signals obtained respectively by the electronic device 500, according to some embodiments of the present disclosure, and the EEG device, according to some comparative embodiments. The first row of FIG. 7B is the waveform of the data signal D4, which is E2-R2, generated by the electronic device 500. The second row of FIG. 7B is the waveform of the comparative O-type signal generated by the comparative EEG device. The comparative O-type signal may be generated by subtracting an EEG signal obtained from the position “O2” of the 10-20 system from another EEG signal obtained from the position “Fpz” of the 10-20 system. The third and fourth rows of FIG. 7B are cross-correlation plot and coherent plot, respectively, of the data signal D4 and the comparative O-type signal. It is also can be observed that the data signal D4 and the comparative O-type signal have good relationship in time domain and in low frequencies.

In the 10-20 system, the positions labeled with “C” and “O” are usually covered with hair; therefore, it is hard to generate high-quality data signals corresponding to these areas. However, according to FIG. 7A and FIG. 7B, the ipsilateral and contralateral mastoid reference electrodes of the wearable electronic device 500, together with multiple differential amplifiers, allow for the simultaneous recording of both ipsilateral and contralateral in-car EEG signals, facilitating extraction of EEG signals that are highly correlated to occipital (positions labeled with “O”) and central (positions labeled with “C”).

Moreover, the electronic device 500 would not experience signal quality deterioration due to the detachment of one of the reference electrodes or the proximity of the sensing electrode to the reference electrode, as the electronic device 500 can pair a sensing electrode with the ipsilateral and contralateral reference electrodes to generate two interchangeable data signals. The controller 509 in some embodiments may monitor the quality of the reference signals. When the controller 509 detects that a reference electrode is partially or fully detached from the skin according to the quality of the reference signal, the controller 509 may use montage-based reconstruction to generate an alternative signal similar to the lost data signal. For example, referring to FIG. 1 and FIG. 5, if the controller 509 detects that the reference electrode 105_1 is partially or fully detached and the data signal D3 is lost, the controller 509 may subtract the data signal D2 (i.e., E1-R2) from the data signal D4 (E2-R2) to generate the alternative signal, which is E2-E1. When the electronic device 500 is worn by the user, the sensing electrode 103_1 is located near the reference electrode 105_1 on the scalp. Therefore, the alternative signal is similar to the lost data signal D3 (i.e., E2-R1).

Furthermore, the electronic device 500 has the capability of real-time edge computing, which not only reduces the burden on the user's mobile device or the cloud infrastructure, but also enables the real-time control of neuromodulation actions (e.g., vibration or microcurrent stimulation or others) based on the extracted features.

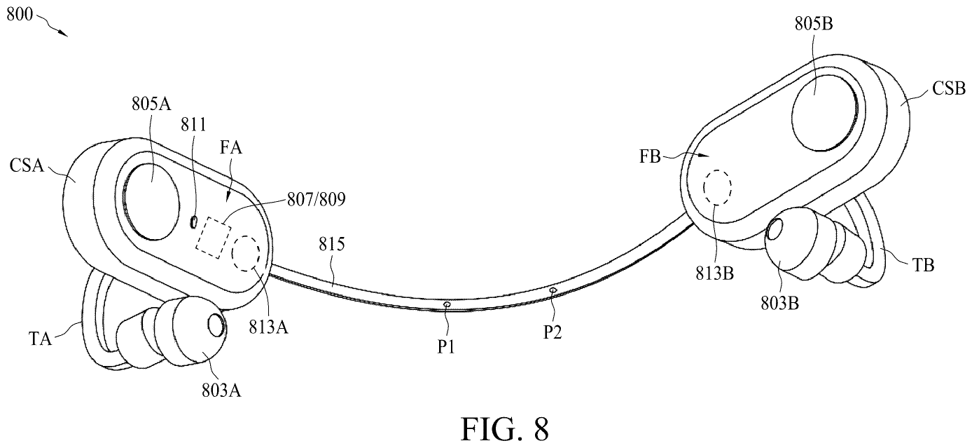

FIG. 8 shows a perspective view of an electronic device 800 according to some embodiments of the present disclosure. The electronic device 800 may be an implementation of the electronic device 500 of FIG. 5. The electronic device 800 includes a pair of sensing electrodes 803A and 803B, a pair of reference electrodes 805A and 805B, and casing parts CSA and CSB. The sensing electrodes 803A and 803B are mounted respectively on the casing parts CSA and CSB. Specifically, the sensing electrode 803A is arranged at a terminal of a protruding portion TA of the casing part CSA. The protruding portion TA have a U-shape, and an opening of the U-shape and a surface FA of the casing part CSA may face substantially in the same direction. The reference electrode 805A is arranged on the surface FA. The casing parts CSA and CSB are of similar shapes. The sensing electrode 803B is arranged at a terminal of a protruding portion TB of the casing part CSB. The reference electrode 805B is arranged on a surface FB of the casing part CSB.

In some embodiments, the sensing electrodes 803A and 803B are made from conductive elastomers shaped like earbuds. The sensing electrodes 803A and 803B may be coated with silver/silver chloride (e.g., Ag/AgCl) to reduce skin impedance, but this disclosure is not limited thereto. The sensing electrodes 803A and 803B are for positioning respectively in the left and right car canals of the user, so as to obtain a pair of physiological signals corresponding respectively to the left and right outer car canals of the user. The reference electrodes 805A and 805B are gel pad snap connectors that may be attached to a pair of gel pads. The reference electrodes 805A and 805B are for connecting respectively with the left and right mastoid processes of the user through the gel pads, so as to obtain a pair of reference signals corresponding respectively to the left and right mastoid processes of the user through the gel pads; however, the present disclosure is not limited thereto. In some embodiments, the reference electrodes 805A and 805B may be contact with regions of the scalp other than the mastoid processes directly or through the gel pads. Accordingly, the sensing electrodes 803A and 803B and the reference electrodes 805A and 805B are arranged to obtain signals measured at positions on the opposite sides of the scalp defined by the midsagittal plane and signals measured at positions on the same side of the scalp defined by the midsagittal plane.

The electronic device 800 further includes a signal processing circuit 807, a controller 809, a PPG sensor 811, a pair of vibration units 813A and 813B, and a connecting line 815. The signal processing circuit 807 and the controller 809 are arranged within the casing part CSA and are electrically coupled to each other. The signal processing circuit 807 is electrically coupled to the sensing electrodes 803A and 803B and the reference electrodes 805A and 805B. The PPG sensor 811 are electrically coupled to the controller 809, and is arranged in the casing part. A light guide element may be arranged on the surface FA adjacent to the reference electrode 805A. The PPG sensor 811 may be arranged at an inner side of the light guide element, so that the surface FA exposes the PPG sensor 811. The PPG sensor 811 is for placing nearby one of the mastoid processes of the user; however, the present disclosure is not limited thereto. The vibration units 813A and 813B are electrically coupled to the controller 809, and are arranged respectively within the casing parts CSA and CSB. The connecting line 815 is coupled between the casing parts CSA and CSB, and is for facilitating the electrical connection of the signal processing circuit 807 and the controller 809 to the other circuit elements. For example, the sensing electrode 803B, the reference electrode 805B, and the vibration unit 813B may be electrically coupled to the signal processing circuit 807 and the controller 809 through the connecting line 815. In some embodiments, the signal processing circuit 807 and/or the controller 809 may be distributed in both the casing parts CSA and CSB, while the separated parts of the signal processing circuit 807 and/or the controller 809 may be electrically connected through the connecting line 815. That is, the connecting line 815 is configured to transmit at least part of the physiological signals and the reference signals generated by the sensing electrodes 803A and 803B and the reference electrodes 805A and 805B.

As can be observed from FIG. 2A, FIG. 2B, FIG. 4, and FIG. 5, the electronic device 800 may include more than a pair of sensing electrodes and more than a pair of reference electrodes. For example, the electronic device 800 may include an additional pair of sensing or reference electrodes mounted respectively on the extension ports P1 and P2 at the connecting line 815. The additional pair of sensing electrodes are for connecting respectively with the left and right sides of the forehead of the user, so as to obtain a pair of physiological signals corresponding respectively to the left and right sides of the forehead of the user.

Since there are needs in the field of neuromodulation, which include (1) lacking of edge-computing devices for personalized treatment strategies based on EEG and other physiological data; (2) lacking of user-friendly monitoring devices for evaluating treatment effectiveness; (3) limited access to AI-assisted brain state analysis; (4) lacking of affordable neuromodulation devices; (5) insufficient personalization capabilities in existing AI systems; and (6) shortage of AI models that can continuously being refined through feedback from the users and healthcare professionals. Therefore, the present disclosure provides a healthcare system 900 shown in FIG. 9.

FIG. 9 is a schematic functional block diagram of the healthcare system 900 according to some embodiments of the present disclosure. The healthcare system 900 includes local components, which include an electronic device 910 for monitoring physiological status and a mobile device 920. The electronic device 910 may be realized by the electronic device 500 of FIG. 5 or the electronic device 800 of FIG. 8. The mobile device 920 may be a smartphone, a tablet, laptop, or other suitable electronic apparatuses. The mobile device 920 is communicatively (e.g., through Bluetooth) coupled to the electronic device 910. The mobile device 920 is configured to receive the analysis result regarding the physiological status of the user from the electronic device 910, and display the analysis result for personal viewing. In some embodiments, the mobile device 920 is an alternative to the electronic device 910 for generating the analysis results based on the physiological signals. The mobile device 920 may receive the physiological signals, such as the EEG signals and/or the PPG signals, from the electronic device 910 for computing the analysis result. The electronic device 910 may perform the neuromodulation actions (e.g., vibration or microcurrent stimulation or others) based on the analysis result generated by and received from the mobile device 920.

In some embodiments, the healthcare system 900 further includes a cloud-based platform, which includes a cloud server 930 and a cloud storage device 940. The cloud storage device 940 is communicatively (e.g., through Internet) coupled to the mobile device 920, in order to automatically sync the physiological signals stored in the mobile device 920 to the cloud storage device 940. In some embodiments, the cloud server 930 is an alternative to the mobile device 920 for generating the analysis result, based on the physiological signals stored in the cloud storage device 940. The electronic device 910 may perform the neuromodulation actions based on the analysis result received from the cloud server 930 via the mobile device 920.

In some embodiments, the cloud storage device 940 further stores a labelled dataset 941 from healthcare professionals, while the user can store feedbacks on his/her subjective experiences during neuromodulation sessions into the cloud storage device 940. The cloud server 930 is configured to execute machine learning models stored in the cloud storage device 940, which can be personalized through user feedbacks and can be updated with the labelled dataset 941. Along with the electronic device 910 used by the user, the machine learning models can generate increasingly personalized analysis reports, which may be transmitted to the mobile device 920 for personal viewing. Therefore, the healthcare system 900 can customize its analysis for the user without compromising the accuracy and clinical validity of the analysis.

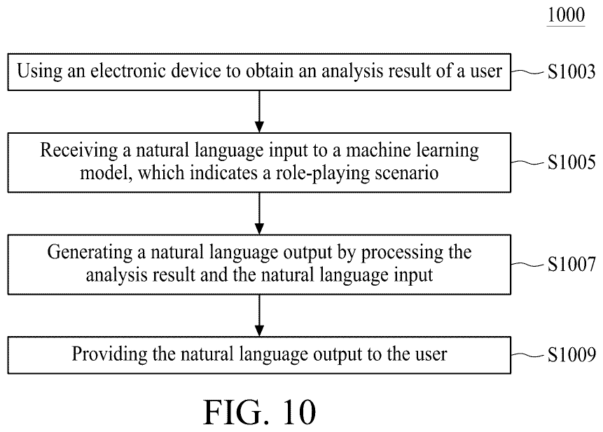

FIG. 10 is a flowchart of a method 1000 for interacting with a user performed by the healthcare system 900, according to some embodiments of the present disclosure. The machine learning models executed by the cloud server 930 may capable of providing responses to the natural language inputs of the user. For example, one of the machine learning models may be a large language model (LLM), such as GPT, Claude, Grok, Llama, Falcon, DeepSeek, etc. When the healthcare system 900 performs the method 1000, the healthcare system 900 can interact with the user to improve his/her physiological status (e.g., anxiety or depression) of the user. In operation S1003, the electronic device 910 obtains the analysis result of the user according to the physiological signals obtained from the user. The analysis result may include, for each channel (e.g., each data signal), the power of brain waves of different frequency bands, such as the energy intensity of one or more frequency bands of gamma wave, beta wave, alpha wave, theta wave, and delta wave. The analysis result may further include asymmetry of brain waves of a predetermined kind (e.g., a predetermined frequency band) in different channels (e.g., different data signals), such as the frontal alpha asymmetry, which is a possible indication of depression. The analysis result may further include the arousal level and/or the emotional valence. Accordingly, the analysis result may include at least one of a physiological data selected from a group consisting of power of brain waves of different frequency bands, asymmetry of brain waves of a predetermined frequency band, the arousal level, and the emotional valence.

In operation S1005, the cloud server 930 receives a natural language input (e.g., prompt) to the machine learning model via, for example, the mobile device 920. The natural language input indicates a role-playing scenario. In some embodiments, the role-playing scenario requires the machine learning model to act as an intelligent character, such as a practitioner of neurofeedback (biofeedback) or a meditation tutor.

In operation S1007, the cloud server 930 processes the analysis result and the natural language input by using the machine learning model (e.g., the LLM) to generate a natural language output. In operation S1009, the cloud server 930 provides the natural language output to the user via the electronic device 910 or the mobile device 920. For example, the natural language output may include the text message or the audio message, and is provided by the display of the mobile device 920 or by the loudspeakers of the mobile device 920 or the electronic device 910. Through the natural language output, the healthcare system 900 may function as a personal trainer teaching or guiding the user in relaxation, meditation, etc., according to the current physiological status of the user. As a result, the physiological status of the user may be effectively improved.

In some embodiments, the electronic device described in the present disclosure can be used to monitor physiological signals such as alpha/theta asymmetry. Alpha asymmetry, as previously mentioned, particularly in the frontal regions, has been consistently linked to depression and anxiety disorders, while theta asymmetry has also been associated with these conditions. A significant proportion of patients with major depressive disorder exhibit resting frontal alpha asymmetry that is not commonly observed in healthy controls. Monitoring alpha/theta asymmetry could provide an objective biomarker for mood disorders, potentially aiding in early detection and predicting treatment responses. Thus, the electronic device in some embodiments of the present disclosure has the capability to measure alpha/theta asymmetry continuously and non-invasively, making it a valuable tool for the longitudinal monitoring of mood disorders.

In some embodiments, the electronic device described in the present disclosure can be used to monitor peak alpha frequency (PAF). PAF is positively correlated with cognitive performance and memory. Individuals with Alzheimer's disease have lower PAF compared to healthy people. Therefore, the electronic device can provide early indicators of cognitive decline or neurodegenerative processes. The PAF captured by the electronic device can also be used to evaluate the efficacy of cognitive enhancement interventions or the efficacy of treatments for neurodegenerative diseases.

In some embodiments, the electronic device described in the present disclosure can be used to monitor flat or suppressed EEG patterns, which can be easily detected especially in the alpha band. The flat or suppressed EEG patterns are related to the epigenetic influences of psychological and/or bio-logical factors, such as increased anxiety, age, increased levels of the corticotrophin releasing factor, steroid hormones, and neuropeptides. Therefore, the electronic device can provide early warnings that signal these risk situations.

In some embodiments, the electronic device described in the present disclosure can be used to monitor alterations in default mode network (DMN) activity. DMN activity have been linked to various neuropsychiatric disorders and pain. For instance, DMN dysfunction occurs in dementia, schizophrenia, epilepsy, anxiety, depression, autism, and attention-deficit/hyperactivity disorder (ADHD). The electronic device in some embodiments has ability to capture idle state EEG, providing insights into DMN function and can potentially aid in the diagnosis and monitoring of various neuropsychiatric conditions. The electronic device can be further used to track changes in DMN activity during or after interventions like mindfulness training or CES, providing objective measures of treatment efficacy.

In some embodiments, the electronic device described in this disclosure further include microphones for capturing audio data of the sleeping user. Multi-modal approaches to sleep staging have shown improved accuracy over single-modality methods. Therefore, the electronic device can improve the diagnosis and monitoring of sleep disorders by integrating EEG, motion detection, PPG, and audio data. In some embodiments, the electronic device can be used to monitor rapid eye movement (REM) sleep behavior disorder (RBD), which is a precursor to neurodegenerative diseases like Parkinson's disease. The electronic device can provide comprehensive data for accurate RBD diagnosis and monitoring, potentially allowing for early intervention in neurodegenerative processes. The electronic device may wake the subject up and acquire clinical history of whether the subject is “acting their dreams” during sleep, which is a predominant criteria for RBD determination.

The associations between the EEG power and the cerebral blood flow measured by (functional magnetic resonance imaging) fMRI during sleep has been demonstrated. The simultaneous measurement of EEG and PPG parameters performed by the electronic device described in this disclosure can provide insights into cerebral autoregulation during sleep, potentially uncovering new biomarkers for neurological conditions.

It has been demonstrated that the massage therapy can alter EEG patterns and resting fMRI connectivity, and also can decrease depression levels in depression subjects. In some embodiments, the electronic device described in this disclosure can provide mechanical afferent touch therapy (MATT) while simultaneously recording EEG signals. Therefore, the electronic device of this disclosure help elucidate the neurophysiological mechanisms underlying the potential therapeutic effects in depression of mechanical stimulation.

Transcutaneous vagus nerve stimulation (tVNS), which is a form of cranial electrotherapy stimulation (CES), is a technique where an electrical current is applied to the vagus nerve. tVNS can increase resting-state functional connectivity in the default mode network and the cognitive control network. In some embodiments, the electronic device described in this disclosure combines tVNS with EEG recording that can monitor brain responses to stimulation in real-time, allowing for personalized optimization of treatment parameters.

In some embodiments, the electronic device of this disclosure provide CES close to the vestibular nerve and the trigeminal nerve while monitoring EEG signals and the user's motion. Galvanic vestibular stimulation has shown promise in treating emotional disorders and insomnia, while external trigeminal nerve stimulation was effective in treating migraine and ADHD symptoms in children. Therefore, the electronic device provides a novel approach for treating and monitoring the efficacy of treatments.

A glymphatic system has been demonstrated to be responsible of drainage of molecules, including B-amyloid, from the brain. Sleep stages affect glymphatic system function. The clearance of B-amyloid during deep sleep in mice is increased. In some embodiments, the electronic device of this disclosure combines the sleep monitoring with the CES that potentially can enhance glymphatic flow. Therefore, the electronic device offers a novel approach to neurodegenerative disease prevention.

Specific EEG patterns can be used to predict the migraine attack. For example, the theta power increases in the pre-ictal phase of the migraine attack. In some embodiments, the electronic device of this disclosure combines the CES with EEG recording, allowing for early detection of pre-ictal state of the migraine attack and also allowing the timely intervention. Therefore, the electronic device can potentially prevent or mitigate the migraine attack.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An electronic device, comprising:
a plurality of sensing electrodes, configured to obtain a first physiological signal and a second physiological signal different from the first physiological signal;
a plurality of reference electrodes, configured to obtain a first reference signal and a second reference signal different from the first reference signal; and
a plurality of amplifiers, coupled to the plurality of sensing electrodes and the plurality of reference electrodes, and configured to:
subtract the first reference signal from the first physiological signal to generate a first data signal;
subtract the second reference signal from the first physiological signal to generate a second data signal;
subtract the first reference signal from the second physiological signal to generate a third data signal; and
subtract the second reference signal from the second physiological signal to generate a fourth data signal.

2. The electronic device of claim 1, wherein the first physiological signal, the second physiological signal, the first reference signal, and the second reference signal are electroencephalography signals.

3. The electronic device of claim 1, wherein a number of the plurality of sensing electrodes is M, a number of the plurality of reference electrodes is N, and a number of the plurality of amplifiers is M×N, wherein M and N are positive integers.

4. The electronic device of claim 1, wherein the plurality of amplifiers comprises a first amplifier, a second amplifier, a third amplifier, and a fourth amplifier, and wherein:
a non-inverting input terminal of the first amplifier and a non-inverting input terminal of the second amplifier are coupled to each other, and are configured to receive the first physiological signal,
a non-inverting input terminal of the third amplifier and a non-inverting input terminal of the fourth amplifier are coupled to each other, and are configured to receive the second physiological signal,
an inverting input terminal of the first amplifier and an inverting input terminal of the third amplifier are coupled to each other, and are configured to receive the first reference signal, and
an inverting input terminal of the second amplifier and an inverting input terminal of the fourth amplifier are coupled to each other, and are configured to receive the second reference signal.

5. The electronic device of claim 4, wherein the plurality of amplifiers further comprises:
a fifth amplifier, wherein a non-inverting input terminal of the fifth amplifier is configured to receive a third physiological signal, an inverting input terminal of the fifth amplifier is configured to receive the first physiological signal, and the output terminal of the fifth amplifier is configured to output a first intermediate data signal; and
a sixth amplifier, wherein a non-inverting input terminal of the sixth amplifier is configured to receive a fourth physiological signal, and an inverting input terminal of the sixth amplifier is configured to receive the second physiological signal, and the output terminal of the sixth amplifier is configured to output a second intermediate data signal,
wherein the electronic device is configured to:
add the first intermediate data signal to the first data signal and the second data signal to obtain a first additional data signal and a second additional data signal; and
add the second intermediate data signal to the third data signal and the fourth data signal to obtain a third additional data signal and a fourth additional data signal.

6. The electronic device of claim 5, wherein the third physiological signal corresponds to a left side of a forehead of a user, and the fourth physiological signal corresponds to a right side of the forehead of the user.

7. The electronic device of claim 1, wherein the plurality of reference electrodes comprise a first electrode and a second electrode configured to obtain the first reference signal and the second reference signal, respectively, wherein the electronic device further comprises:
a first resistor coupled between the first electrode and a ground terminal within the electronic device; and
a second resistor coupled between the second electrode and the ground terminal.

8. The electronic device of claim 1, wherein the first physiological signal corresponds to a left outer ear canal of a user, and the second physiological signal corresponds to a right outer ear canal of the user.

9. The electronic device of claim 1, wherein the first reference signal corresponds to a left mastoid process of a user, and the second reference signal corresponds to a right mastoid process of the user.

10. An electronic device, comprising:
a first pair of sensing electrodes, configured to obtain a first physiological signal and a second physiological signal corresponding to outer ear canals of a user;
a pair of reference electrodes, configured to obtain a first reference signal and a second reference signal corresponding to mastoid processes of the user; and
a controller, coupled to the first pair of sensing electrodes and the pair of reference electrodes, and configured to generate an analysis result according to (1) differences between the first physiological signal and each of the first reference signal and the second reference signal and (2) differences between the second physiological signal and each of the first reference signal and the second reference signal.

11. The electronic device of claim 10, wherein the first pair of sensing electrodes and the pair of reference electrodes are arranged to obtain a plurality of signals measured on opposite sides of a scalp and a plurality of signals measured on the same side of the scalp.

12. The electronic device of claim 10, further comprising:
a first casing part;
a second casing part, wherein the first pair of sensing electrodes are mounted respectively on the first casing part and the second casing part, and the pair of reference electrodes are mounted respectively on the first casing part and the second casing part; and
a connecting line, coupled between the first casing part and the second casing part, and configured to transmit at least part of the first physiological signal, the second physiological signal, the first reference signal, and the second reference signal.

13. The electronic device of claim 12, further comprising:
a photoplethysmography (PPG) sensor, arranged in the first casing part, and configured to obtain a PPG signal corresponding to one of the mastoid processes of the user or corresponding to an area near the one of the mastoid processes,
wherein the control circuit is further configured to generate the analysis result according to the PPG signal.

14. The electronic device of claim 12, further comprising:
a pair of vibration units, respectively arranged in the first casing part and the second casing part, wherein the controller is further configured to control vibration intensities, waveform, or frequencies of the pair of vibration units according to the analysis result or user inputs.

15. The electronic device of claim 10, wherein the pair of reference electrodes are configured to obtain the first reference signal and the second reference signal through a pair of gel pads or configured to directly contact with the scalp to obtain the first reference signal and the second reference signal.

16. The electronic device of claim 10, wherein the pair of reference electrodes are connected to a ground terminal within the electronic device through a pair of resistors.

17. The electronic device of claim 10, further comprising:
a second pair of sensing electrodes, configured to obtain a third physiological signal and a fourth physiological signal corresponding to a left side and a right side of a forehead of the user; and
a signal processing circuit, configured to:
obtain a first data signal and a second data signal according to the differences between the first physiological signal and each of the first reference signal and the second reference signal;
obtain a third data signal and a fourth data signal according to the differences between the second physiological signal and each of the first reference signal and the second reference signal;
obtain a first intermediate signal according to a difference between the third physiological signal and the first physiological signal;
obtain a second intermediate signal according to a difference between the fourth physiological signal and the second physiological signal;
obtain a first additional data signal and a second additional data signal by adding the first intermediate data signal to the first data signal and the second data signal; and
obtain a third additional data signal and a fourth additional data signal by adding the second intermediate data signal to the third data signal and the fourth data signal,
wherein the controller is configured to generate the analysis result according to the first through fourth data signals and the first through fourth additional data signals.

18. A method for processing physiological data, comprising:
using the electronic device of claim 10 to obtain the analysis result;
receiving a natural language input to a machine learning model, wherein the natural language input indicates a role-playing scenario;
generating a natural language output by processing the analysis result and the natural language input by using the machine learning model; and
providing the natural language output,
wherein the analysis result comprises at least one of a physiological data selected from a group consisting of power of brain waves of different frequency bands, asymmetry of brain waves of a predetermined frequency band, an arousal level, and an emotional valence.

19. A healthcare system, comprising:
the electronic device of claim 10, configured to obtain the analysis result; and
a cloud server, configured to execute a machine learning model, and further configured to:
receive a natural language input to the machine learning model, wherein the natural language input indicates a role-playing scenario;
generate a natural language output by processing the analysis result and the natural language input by using the machine learning model; and
provide the natural language output,
wherein the analysis result comprises at least one of a physiological data selected from a group consisting of power of brain waves of different frequency bands, asymmetry of brain waves of a predetermined frequency band, an arousal level, and an emotional valence.

20. A method for monitoring physiological signals, comprising:
obtaining a first physiological signal and a second physiological signal different from the first physiological signal;
obtaining a first reference signal and a second reference signal different from the first reference signal; and
generating an analysis result by (1) comparing the first physiological signal with each of the first reference signal and the second reference signal and (2) comparing the second physiological signal with each of the first reference signal and the second reference signal,
wherein the first physiological signal and the second physiological signal are obtained by a pair of sensing electrodes arranged on opposite sides of a scalp, and the first reference signal and the second reference signal are obtained by a pair of reference electrodes arranged on the opposite sides of the scalp.

21. The method of claim 20, wherein generating the analysis result comprising:
subtracting the first reference signal from the first physiological signal to generate a first data signal;
subtracting the second reference signal from the first physiological signal to generate a second data signal;
subtracting the first reference signal from the second physiological signal to generate a third data signal;
subtracting a second reference signal from the second physiological signal to generate a fourth data signal; and
generating the analysis result according to the first data signal, the second data signal, the third data signal, and the fourth data signal.

22. The method of claim 20, further comprising: determining a sleep stage based on the analysis result generated based on the first data signal, the second data signal, the third data signal, the fourth data signal, a motional signal, and PPG signals.
